(19) United States
(12) Patent Application Publication
(10) Pub. No.: US 2008/0269616 A1
(43) Pub. Date: Oct. 30, 2008

(54) MIR SPECTROSCOPY OF TISSUE

(76) Inventors: Matthew M. Bloom, Menlo Park, CA (US); Bernhard B. Sterling, Danville, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/941,816

(22) Filed: Nov. 16, 2007

Related U.S. Application Data
(60) Provisional application No. 60/866,407, filed on Nov. 17, 2006.

(51) Int. Cl. A61B 5/1455

(52) U.S. Cl. 600/475

(57) ABSTRACT

Disclosed are methods of determining long-term deposition pattern of a compound in tissue. The following steps can be followed: placing tissue against a receptor; directing mid-infrared electromagnetic radiation onto the tissue; quantifying the electromagnetic radiation that is reflected from the tissue to obtain a reflected amount; using a calibration equation to calculate the concentration of a compound from the reflected amount; and using the concentration of the compound to evaluate presence of a clinical condition in the tissue.

### PRIORITY INFORMATION

This application claims the benefit under 35 U.S.C. § 119(e) of U.S. Provisional Patent Application No. 60/866,407, filed Nov. 17, 2006 (attorney docket no. MBLOM.002PR). The entirety of the above-referenced application is hereby incorporated by reference and made part of this specification.

### BACKGROUND

1. Field

The present disclosure relates to measurement of the concentration of a compound in the skin of a subject, for example a human or animal. For example, in some embodiments, a method can be used to determine a concentration of a compound in the skin, and optionally, to correlate the measured concentration of the compound to a specific clinical condition or to the propensity for a specific clinical condition. Mid Infrared spectroscopy can be used to interrogate the tissue.

2. Description of the Related Art

In U.S. Pat. No. 6,365,363, Parfenov et al. describe a method of indirectly measuring the concentration of cholesterol in the skin of a subject by enzymatically oxidizing the sterol in a section of the subject's skin and then quantitating the amount of the hydrogen peroxide by-product stoichiometrically formed in this reaction using a second enzymatic reaction. As a complex series of enzymatic reactions are used in this method to indirectly determine the concentration of cholesterol, the method is both costly and prone to error. In addition, the development of a result using this method is time consuming, and is non-specific for all skin sterol species.

In U.S. Pat. Nos. 6,236,047 and 6,040,578, Malin et al. describe a method for determining the concentration of a blood compound using light in the near-infrared range by analyzing diffusively reflecting radiation emerging from the irradiated sample. However, there is no teaching in these patents as to the determination of concentrations of constituents in the skin of a subject.

Hall et al. also describe in U.S. Pat. No. 5,361,758 a non-invasive technique for directly measuring the concentration of constituents of blood using light in the near-infrared range. No specific methods for the determination of compounds within skin are provided.

### SUMMARY

The following example embodiments are non-limiting. Some embodiments comprise a method of accurately measuring body cholesterol levels without inaccuracies related to recent diet. For example, the method can comprise: irradiating skin tissue with mid-infrared radiation to excite lipid molecules within superficial layers of the tissue; measuring resulting mid-infrared radiation; comparing the measured radiation to stored reference data to obtain correlation information; and using the correlation information to determine body lipid levels without inaccuracies related to recent diet. Resulting mid-infrared radiation can comprise reflected and or transmitted radiation. The method can be used to excite all cholesterol molecules within superficial tissue layers, to excite esterified cholesterol, to excite free cholesterol, to excite free fatty acids, to excite ceramides, etc. The method can comprise assessing a disease, diagnosing psoriasis, or assess risk of cardiovascular disease, for example.

Some embodiments comprise a method of determining a living body's long-term deposition pattern of a compound. For example, the method can comprise: placing skin of a living body against a receptor; directing mid-infrared electromagnetic radiation onto the skin of the living body; quantifying the electromagnetic radiation that is reflected from the skin to obtain a reflected amount; using a calibration equation to calculate the concentration of a compound from the reflected amount; and using the concentration of the compound to evaluate risk of a clinical condition. Using a calibration equation can comprise using a least-squares best fit statistical comparison. The method can comprise evaluating risk of cardiovascular disease. The method can comprise calculating the concentration of a lipid (e.g., cholesterol) from the reflected amount. The method can use an algorithm to indicate a correlation between amount of the compound and any of the following: presence of a medical condition; severity of a medical condition; risk of a developing a medical condition; a prediction of success of a treatment for a medical condition; and/or documentation of success of a treatment for a medical condition.

Some embodiments comprise a method of testing for a type of cholesterol to diagnose a clinical condition. For example, the method can comprise: preparing tissue; placing a probe in proximity to tissue; irradiating tissue with infrared radiation, thereby exciting molecules of a species in the tissue; collecting resulting information from those molecules to determine a concentration of molecules of that species within the tissue; and correlating the concentration to a clinical condition. Preparing can comprise scraping and/or cleaning, for example. Placing a probe in proximity to tissue can comprise contacting the probe to the tissue. The tissue can be skin tissue. The infrared radiation can be mid-infrared radiation. Correlating the concentration to a clinical condition can comprise correlating the concentration to a propensity for a clinical condition. The tissue in the method can be skin, and the method can further comprise all of the same steps performed at least a second time on a second tissue portion located deeper in a subject's skin. The method can further comprise all of the same steps performed at least a second time on a second tissue portion located on the same skin level. The method steps can be performed one time on skin affected by a dermatological condition and another time on skin unaffected by the dermatological condition.

Some embodiments comprise a spectroscopic method of measuring one or more cholesterol species in tissue. For example, the method can comprise: selecting one or more mid-infrared radiation wavelengths to provide information about the one or more cholesterol species; irradiating tissue with the one or more mid-infrared wavelengths; measuring non-absorbed radiation; calculating, using the measured radiation to determine quantities of the one or more cholesterol species in the tissue; and storing the result of the calculation in a computer-readable medium. The method can comprise identifying at least two cholesterol species. Wavelengths can be selected to identify non-free cholesterol. Calculating can comprise comparing the quantity of one species of cholesterol to the quantity of another species of cholesterol. Calculating can comprise taking a ratio between data from one wavelength to the data from another wavelength. The method can comprise quantifying free, esterified, and total cholesterol by taking measurements of only two of the three cholesterol species.

Some embodiments comprise a method of testing for dermatological disease. For example, the method can comprise: providing a source of infrared radiation; providing a dermatological sample; directing the infrared radiation to illuminate the dermatological sample; detecting radiation reflected from the dermatological sample; and using the detected radiation to calculate concentration of an analyte related to a dermatological disease to determine a disease status of the sample. The source of infrared radiation can emit mid-infrared radiation. Using the detected radiation to calculate concentration of an analyte related to a dermatological disease can comprise one or more of the following: calculating concentration of total cholesterol; calculating concentration of free cholesterol; calculating concentration of esterified cholesterol; calculating concentration of free fatty acids; calculating concentration of ceramides; and/or calculating concentration of an analyte related to psoriasis. Determining a disease status of a sample can comprise: diagnosing a sample; grading the severity of a sample; predicting further outbreak of the disease; predicting success of a treatment of the disease; and/or quantifying the success of treatments.

Some embodiments comprise an apparatus for determining how effectively skin absorbs medication. For example, the method can comprise: a mid-infrared radiation source; a skin holder; a radiation detector configured to detect and transmit radiation information in response to mid-infrared radiation impinging on the detector; a radiation path from the radiation source to the skin to the radiation detector; a processor configured to receive data from the radiation detector and determine, from the data, quantities of medication in the skin. The processor can be further configured to determine quantitative information regarding esterified tissue cholesterol.

Some embodiments comprise a method of diagnosing a systemic condition. For example, the method can comprise: irradiating a dermatological sample with mid-infrared radiation; measuring non-absorbed radiation; calculating, from the non-absorbed radiation, how much radiation was absorbed to determine a quantity of an absorbing substance in the dermatological sample; and correlating the amount of the absorbing substance in the dermatological sample to diagnose a systemic condition. Correlating the amount of the absorbing substance in the dermatological sample to diagnose a systemic condition can comprise: diagnosing body hydration or intravascular volume; correlating the amount of glycosylated products in the dermatological sample to determine a systemic blood sugar level trend; diagnosing inflammation and/or infection related to a wound; correlating the amount of a glycosylated species in the dermatological sample to diagnose and/or evaluate treatment prognosis for diabetic disease; correlating the amount of cholesterol and/or phospholipids in the dermatological sample to diagnose diabetes; correlating the amount of bacterial species in the dermatological sample to assess their effects on the progress of wound healing; correlating the amount of bacterial species in the dermatological sample to determine the permeability of the dermatological sample to topically applied substances; correlating the amount of bacterial species in the dermatological sample to determine the susceptibility of the dermatological sample to pharmacological compounds; assessing cancerous or pre-cancerous tissue; assessing aging skin characteristics; correlating the accumulated products of metabolism errors (e.g., PKU, bilirubin) in the dermatological sample to diagnose a systemic condition; correlating the accumulated products of toxic exposure in the dermatological sample (e.g., phenols) to assess exposure of the dermatological sample; quantifying the accumulated products of illicit drug use (e.g., THC) in the dermatological sample; correlating the amount of the absorbing substance in hair and/or nails.

### BRIEF DESCRIPTION OF THE DRAWINGS

The following drawings and the associated descriptions are provided to illustrate embodiments of the present disclosure and do not limit the scope of the claims.

FIG. 1 shows data obtained using a spectrophotometer measuring 5% and 10% preparations of cholesterol in oil;

FIG. 2 shows a close up of a portion of the data of FIG. 1;

### DETAILED DESCRIPTION

Although certain preferred embodiments and examples are disclosed below, inventive subject matter extends beyond the specifically disclosed embodiments to other alternative embodiments and/or uses of the invention, and to modifications and equivalents thereof. Thus, the scope of the inventions herein disclosed is not limited by any of the particular embodiments described below. For example, in any method or process disclosed herein, the acts or operations of the method or process may be performed in any suitable sequence and are not necessarily limited to any particular disclosed sequence. For purposes of contrasting various embodiments with the prior art, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein. The systems and methods discussed herein can be used anywhere, including, for example, in laboratories, hospitals, healthcare facilities, intensive care units (ICUs), or residences. Moreover, the systems and methods discussed herein can be used for invasive techniques, as well as non-invasive techniques or techniques that do not involve a body or a patient.

### Value of Skin Cholesterol Tests

The skin's content of cholesterol may be an independent marker of risk of cardiovascular disease, in addition to, or in place of, the conventional markers of cardiovascular risk such as serum LDL level, LDL/HDL or (Total Cholesterol)/HDL ratios, Framingham risk assessment, etc. Skin cholesterol levels do not correlate with serum levels (i.e., circulating HDL, LDL, VLDL cholesterol species), and accordingly, these tests may represent a new way to risk assess, or to further risk assess, patients. In fact, it is proposed that measuring the cholesterol content of tissue likely represents a ‘functional test’ of how the body processes cholesterol (both cholesterol directly from the diet, and cholesterol mobilized from storage in the liver) and deposits it elsewhere, in locations that cause morbidity, such as in the walls of blood vessels. Indeed, there is likely an overall, longer time constant, global rate at which or pattern with which the body deposits cholesterol into tissues, which is desirably less sensitive to recent diet and other confounding events than are current techniques of cholesterol measurement such as serum sampling.

Thus, measurement of skin cholesterol levels likely represents a more accurate and meaningful parameter than measuring serum cholesterol levels. There is clinical evidence that there is a relationship between what is deposited into blood vessels and what is deposited into other tissues such as skin. Measurement of skin cholesterol is furthermore proposed as a means to determine the rate at which skin tissue synthesizes cholesterol species from precursors and/or the rate at which cholesterol is transported from the blood and deposited into tissue. This may be tied to the differences between tissue cholesterol pools, in its free and esterified forms.

A skin cholesterol measurement can be accomplished independently or separately from traditional risk assessment methods, and it may have distinct or additive prognostic value. A skin cholesterol measurement can aid in further risk-stratifying patients for vascular disease (e.g., cardiovascular, cerebrovascular, peripheral vascular, etc.).

Measuring skin cholesterol with mid infrared (Mid IR) radiation has some distinct advantages: 1) no blood sample needed; 2) painless; 3) quick to perform; 4) no chemicals applied to skin; 5) results available quickly; 6) no need for patients to fast the night before; 7) reproducible; 8) may be a new marker for disease risk; 9) amenable to mass screenings and outpatient settings; 10) can detect and quantify the total skin cholesterol, as well as the individual subspecies of free and esterified tissue cholesterol, which generally cannot be done with digitonin binding methods which nonspecifically react with all forms of free sterols.

### Mid Infra-Red

The use of Mid IR wavelengths offers some unique improvements on methods described by others. For example, Mid IR has a short penetration depth. Therefore, it interrogates only the cell layers nearest the probe tip. In skin, this cell layer is the stratum corneum. Because the stratum corneum is avascular, the Mid IR signal that is being received has not come in contact with blood, and would generally not contain information from species in circulation. This is different from Near IR systems, which generally would contain information from species in circulation. Therefore any species that is observed, such as cholesterol, would generally not have a substantial blood-based (intravascular) component. Likewise, Mid IR is well suited to examining the contents of the extravascular spaces, which include intercellular, as well as interstitial fluid spaces, something Near IR cannot readily do. In the case of tissue cholesterol, this characteristic of Mid IR interrogation means that a signal obtained from skin does not include information relating to serum cholesterol species, such as HDL, LDL, VLDL, etc, which are forms of cholesterol bound to High Density proteins, Low Density proteins, Very Low Density proteins, etc. (The cholesterol portion makes the molecule a “Lipoprotein,” which explains the use of the “L” in High-density lipoprotein (HDL)). Instead, in tissue, the cholesterol is mainly in the form of cholesterol (also called free cholesterol), or in the form of a cholesterol ester.

An additional benefit of using Mid IR is that because it excites molecules at their fundamental frequencies, it allows for the precise identification of species. Furthermore, Mid IR has a signal intensity that is orders of magnitude greater than that of Near IR. In contrast, Near IR measures the upper harmonics of the resonating frequencies, which are orders of magnitude weaker and can overlap between different entities. The specificity of MID IR allows for the identification of precise species, e.g., quantification of free and esterified cholesterols, as well as total cholesterol pools. The ability to determine any or all of total, free, and esterified, cholesterol species is an advantage over those means which identify only free skin sterol (e.g., chemical tests, such as those that employ digitonin).

In some embodiments, clinically relevant information can be found in the ratios or absolute quantities of these species with respect to each other, or with respect to other entities. These quantities may be derived by spectroscopic means, including individual species of sterol subspecies, or other spectroscopically obtained parameters, or they may be obtained via separate methods. Such methods can include serum tests or other clinical values or questionnaire results, such as a Framingham risk evaluation. In total, these methods can provide meaningful clinical information.

Changes in concentration of total cholesterol, free cholesterol, esterified cholesterol, free fatty acids, ceramides, etc, are related to dermatologic diseases such as psoriasis. The ability to quantify these individual species is proposed as the basis of a separate series of tests for dermatologic diseases, to diagnose, grade the severity of, predict further outbreak of, or quantify the success of treatments.

Cholesterol content of skin directly affects the permeability of the skin to the passage of various substances, both into, and out of, the body. This information is proposed as a marker of the utility of pharmacological preparations designed either to primarily enhance the health of skin tissue, or of the success of preparations designed to permeate through the skin (as a route of delivery) and have more systemic effects (e.g., drug delivery through the skin). Gathering information about skin cholesterol species pools in general, and esterified cholesterol in particular, can likely allow determination of how effectively a patients' skin would be able to absorb medication. The determination of esterified tissue cholesterol can not be performed using digitonin binding techniques, which quantify free sterols.

Additionally, use of Mid IR can lead to new information being gained about other systemic diseases, in addition to cholesterol deposition, which have a dermatologic component and may be diagnosed or evaluated. For example, skin hydration can be a marker of overall body hydration or intravascular volume. Glycosylated products can be a marker of systemic blood sugar levels. Additional markers may be available to indicate inflammation/infection in the areas of wounds. It is proposed that the quantification of glycosylated species such as proteins or lipid species including cholesterol and fatty acids, can serve as a marker of the severity of diabetic disease, or the inadequacy of its long-term treatment. It has been shown that in diabetic patients, the cholesterol content of the liver is markedly diminished, while the content of cholesterol and phospholipid in skin is greatly increased. This can allow for a non-invasive measure of diabetic severity, and patient medication compliance. Furthermore, the products of infection can be assessed with respect to how those products affect the skin. Additionally, specific bacterial species in the skin can be further characterized, and their effects on the progress of wound healing can be assessed. Additionally, cancerous, or pre-cancerous tissues can be identified and assessed. The characteristics of aging skin can be quantified and analyzed. Additionally, the identification of accumulation products of errors of metabolism (e.g., PKU and bilirubin) such as is performed in newborn screening, is proposed for use with other patients. Accumulated products of toxic exposure (e.g., phenols) can also be measured in the skin, and such a measurement can be useful in mass casualty assessments or occupation exposure settings. Accumulated products of illicit drug use (e.g., THC) can be accomplished rapidly using similar techniques.

Interrogating tissue using Mid Infrared Spectroscopy allows for the quantification of specific species. As outer epidermis is composed of mostly ‘dead’, non-metabolic tissue, information obtained from their examination can reveal insights into the end products of metabolism. For example, the glycosylated products of proteins or lipids, such as cholesterol, can be a marker of systemic blood sugar levels, or of abnormal metabolic processes, such as diabetes. The tissue can also provide important clinical information regarding the functional processing of entities by the body, as the end products of their metabolism, or lack thereof, may be deposited in the skin and can be queried. These entities may be endogenous materials, or drugs or preparations administered through standard routes, i.e., orally, intravenously, intramuscularly, inhaled, applied to skin, etc. The determination of concentrations of specific entities, allowed by Mid Infrared Spectroscopy, allows for the determination of prior exposure to these or metabolic products of these entities, or determination of the normal or abnormal metabolic processing of these or related entities. Similarly, minimally metabolically active tissue such as hair or nails can have trapped products of metabolism, and this tissue can provide a record of the body's exposure to, or accumulation of, various chemical species. The specificity of Mid infrared spectroscopy can detect and quantify these species. The examination of more metabolically active tissue, such as dermis or organs, can reveal metabolic processes in action, and serial examinations over time can provide a picture of the rate at which these metabolic processes occur.

When different tissues are placed against the probe, either in their native state or after pre-conditioning with biological markers or dyes, this device may be used to differentiate ‘healthy’ from ‘unhealthy’ tissue, or normal from malignant or pre-malignant tissue, or be able to grade the degree of health of a tissue along a continuum, such as normal to malignant tissue, healthy from metabolically unhealthy, etc.

The tissue being interrogated may be cleaned with gentle cleansers to remove surface oils, etc., or may be gently scrubbed to remove the outermost layers of tissue. The radiation (e.g., Mid IR radiation) can penetrate to various depths, so that various tissues may be interrogated. Advantageously, the stratum corneum can be interrogated by the signal from the probe. In some cases, however, the signal may penetrate deeper. The epidermis is as thin as 60 microns or less in some individuals. In some cases, the stratum corneum may be as thin as 10 microns, especially when the loose outer layers are removed. The Mid IR may interrogate tissue throughout various layers beyond the stratum corneum alone. Optical probing can be especially effective in gathering information from layers closer to the surface, but deeper epidermal layers can also provide information. For example, the outermost layers can be scraped away, either in cleaning or in preparation, and a signal can be obtained from slightly deeper epidermal layers. In some embodiments, clinically important information may be obtained from depth profiling of the skin, as sequential layers of the epidermis are removed and serial measurements are taken and/or compared.

### Experimental Rationale and Data

The epidermis is a stratified and cornifying epithelium comprising five layers. It varies in thickness from 0.003 to 0.12 mm, except on the palms and soles where it may be 0.8 and 1.4 mm thick, respectively. Epidermal cells are reported to be completely renewed over a period of roughly 28 days. The stratum corneum, or horny layer thickness is 13-15 microns on average. On the palms and soles, this layer attains a thickness of 600 microns. The time it takes for complete renewal of this layer has been variously reported to range from 3 to 13 days.

Published values of the amount of cholesterol in skin, taken from biopsy specimens, when stated on a dry tissue weight basis, are in the range of 1.4-7.2 μg/mg tissue; most reports fall in the range of between 5.0-6.2 μg/mg of cholesterol per mg of dry tissue. Further, reported values of cholesterol, expressed as percentage of total skin lipids, range from 2.6 to 16%.

Comparisons of skin cholesterol levels taken from known groups of ‘normal’ and ‘atherosclerosis’ patients demonstrate levels of total skin cholesterol to be different.

Total skin cholesterol (μg/mg dry weight)

| Authors | Normal | Atherosclerosis |
|---|---|---|
| Melico-Silvestre (1981) | 1.4 | 2.4-3.6 |
| De Graeve (1984) | 4.5 | 5.25 |
| Bouissou (1974) | 4.44 | 5.94 |
| Nikitin (1987) | 7.2 | 9.2 |
| Beaumont (1982) | 1.7 | 2.7 |
| Bouissou (1982) | 4.56 | 5.46 |

This suggests that in patients with atherosclerosis, their skin cholesterol may be increased on the order of 20 to >100% over normal. This allows a calculation of the precision that may be required for detection of a disease state. Assuming that live skin is roughly 50% hydrated, then at a resolution of 1/30: 0.1% total cholesterol×1.4 for diseased patients× 1/30=0.005%. Likewise, 0.1% total cholesterol×1.0 for normal patients× 1/30=0.003%. Accordingly, in some embodiments, one would need a precision of roughly 0.002%, or 20 ppm. Therefore, in some embodiments, a sensitivity of 20 ppm is preferred to allow detection of the cholesterol species in the outer skin at the concentrations through which these species are known to range.

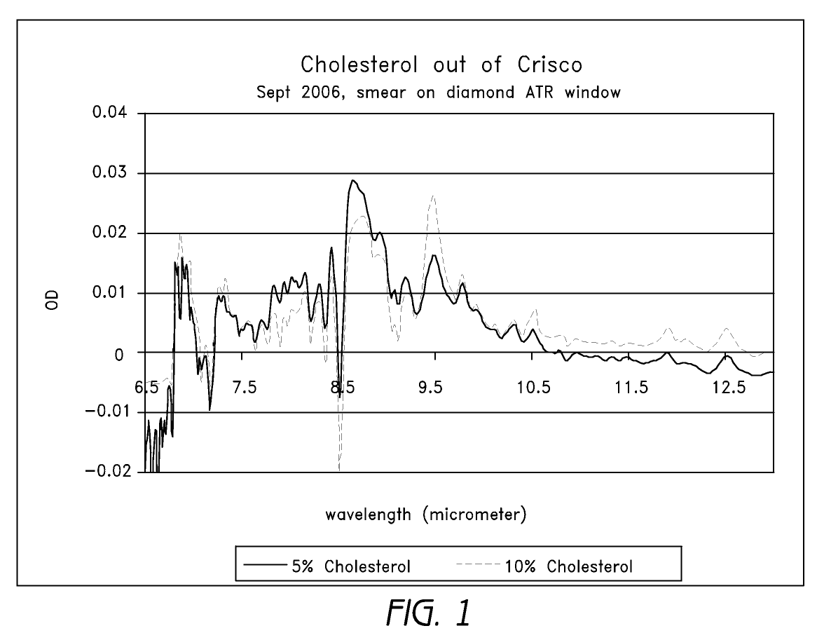

MIR spectroscopy can be used to detect changes in cholesterol concentration, when cholesterol is suspended in a lipid medium. In this case, Crisco oil was used to simulate the lipid environment of the skin. Two separate preparations, one for 5% cholesterol suspended in Crisco, the second, 10% cholesterol in Crisco, were made by dissolution in chloroform and allowed to air dry. The measurements of these two preparations are shown in FIG. 1. The 5% preparation corresponds to the solid line, and the 10% corresponds to the dashed line. The measurements illustrated in FIG. 1 were obtained on a Bruker Series 70 spectrophotometer with a diamond ATR accessory. These measurements demonstrate a measurable difference between the preparations.

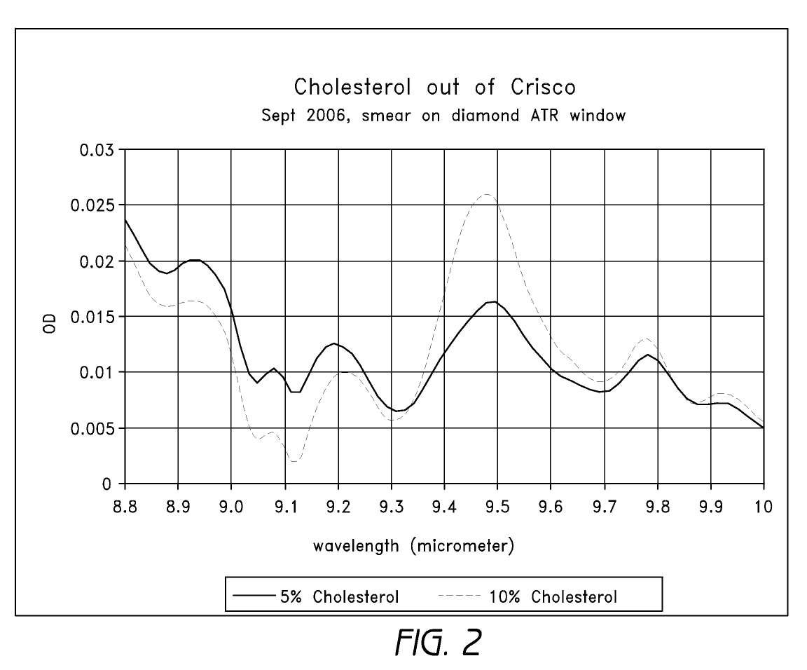

FIG. 2 shows a close-up view of a portion of the data in FIG. 1. As shown, further inspection of the region between roughly 9.4 to 9.58 demonstrates that a measurable difference can be seen around a wavelength of 9.5 microns (wavenumber 1052 cm⁻¹). This corresponds to published peaks for cholesterol at around wavenumber 1057 cm⁻¹. All named frequencies are approximate, and can vary as much as +/−20 cm⁻¹ depending on specific spectroscopic and material conditions.

In the some embodiments, mid-infrared spectra (4000-400 cm⁻¹) of tissue samples are acquired and quantitative information extracted using spectral features or patterns in the ranges 900-1500 cm⁻¹, 1500-1800 cm⁻¹, and 2800-3200 cm⁻¹. In particular, the data is analyzed to determine the levels of total cholesterol, free cholesterol, and esterified cholesterol. Other species such as fatty acids, triglycerides, total lipids, phospholipids, etc. can be similarly quantified.

The dominant spectra of other species of interest are known to those learned in the art. Cholesterol demonstrates dominant bands at 1057, 1466, 1381 cm⁻¹. Cholesterol ester demonstrates dominant bands at 1740, 1466, 1381, 1170 cm⁻¹. Triglycerides demonstrate dominant bands 1736, 1474, 1180 cm⁻¹. All of these named compounds have multiple lesser bands which may be utilized as well.

From the mid-infrared spectra, one can gather general information concerning the molecular constituents and their structures. For instance, there are two prominent amide absorptions, one at approximately 1655 cm⁻¹, (arising from C═O stretching, and termed the amide I band) and another at approximately 1550 cm⁻¹, originating from N—H bending (termed the amide II band) vibrations of the peptide groups in proteins. The sharp absorption at 1467 cm⁻¹ is attributed to the bending (scissoring) vibrations of the CH2 groups of the lipid acyl chains, with the shoulder at 1446 cm⁻¹ arising from the asymmetric bending vibration of the CH3 groups of both lipid and protein constituents. The CH3 symmetric bending vibration gives rise to the absorption at 1378 cm⁻¹. Absorptions at approximately 1242 and 1088 cm⁻¹ come from the PO2- asymmetric and symmetric stretching vibrations of the phosphodiester groups of phospholipids. The remaining absorptions originate from ester C-0-C asymmetric and symmetric stretching vibrations (approximately 1173 and 1065 cm⁻¹ respectively) of phospholipids, triglycerides and cholesterol esters. The most prominent lipid absorption is that in the esterified cholesterol spectrum at approximately 1740 cm⁻¹, arising predominantly from the ester C═O groups of cholesterol esters, while the strong bands at approximately 2852 and 2926 cm⁻¹ originate with the symmetric and asymmetric stretching vibrations of the lipid acyl CH2 groups.

The fact that the spectra of esterified and free cholesterols are clearly different from one another supports the notion that esterified and free cholesterol can be quantified separately, based upon IR spectroscopy of tissue.

In some embodiments, all three categories of cholesterol (total cholesterol, free cholesterol and esterified cholesterol) may be quantified from determinations of any combination of two of these species. For example, because these categories are related by the following equation: total cholesterol=free cholesterol+esterified cholesterol, esterified cholesterol can be inferred from knowledge of total and free cholesterol.

Water spectra dominates in mammalian tissues, and may need to be subtracted from the experimental spectrum for more optimal analysis.

While the ATR method is well suited to in vivo sampling and to accurate subtraction of the water signal, spectra collected with the ATR method are not equivalent to IR absorption spectra, but depend on properties of the ATR material and the sample, in addition to the sample absorption coefficient. For instance, the penetration depth of the evanescent sampling wave depends on the refractive indices of the ATR material and the sample. In addition, the varied affinities for the ATR material of different moieties in the tissue may play an important role in the intensities of the observed bands.

Additional mathematical processing can be used to correct for the nonlinear baseline shifts seen at this magnification. The results of this investigation suggest that one can detect differences in cholesterol concentrations using MIR imaging techniques, against a background of other lipids, in the concentrations in which they appear in vivo in epidermal tissue.

All named frequencies and/or wavelengths discussed herein are approximate, and can vary by amounts understood by those of skill in the art. For example, frequencies can vary by plus or minus 15 cm⁻¹. Variation can depend, in some embodiments on specific spectroscopic and/or material conditions.

Some embodiments provide an apparatus and a method for identifying the risk of a clinical condition in a human or animal by correlating Mid Infrared (MIR) absorbance spectral data with one or several parameters including a concentration of one or more substances in the skin, a score that can be derived from one or more clinical tests like a stress test on a treadmill, coronary angiography, or intravascular coronary ultrasound. The method determines the concentration of a compound in the skin of a human or animal, and it can comprise the steps of placing a part of the skin against a receptor, directing electromagnetic radiation (EMR) from the mid-infrared spectrum onto the skin, measuring a quantity of EMR reflected by, or transmitted through, the skin with a detector; and performing a quantitative mathematical analysis of the quantity of EMR to determine the concentration of the compound, for example free and esterified cholesterol. An example of a clinical condition is cardiovascular disease.

“Normal” concentrations of species such as cholesterol pools can vary as a function of a subject's age, race, gender, etc. This patient-specific data may be used to normalize the collected data, or used to more accurately distinguish “normal” versus “abnormal” medical conditions.

The concentration of certain compounds in the skin of a subject may be used to assess the risk of development or the severity of specific medical conditions in that subject. Early detection of these types of risks in a patient permits measures to be taken that may slow or even prevent the onset of these conditions. As an example, it has been determined that elevated concentrations of cholesterol in the skin of an individual is an indication of a risk for cardiovascular disease. Therefore, the development of simple, non-invasive methods for determining the concentration of skin compounds is of importance. Examples of other compounds that can be advantageously measured includes fats, proteins, including cell-surface proteins, glycoproteins, lipoproteins, carbohydrates, and steroids, ceramides, and glycosylated lipids, (e.g., glycosylated sterol). In some preferred embodiments, the compound to be measured is preferably a steroid such as cholesterol.

Some embodiments use a correlation step to relate the measurements of transmitted or reflected light to a concentration value for one or more than one given compounds. If desired, the measured concentration of the compound may be related to a particular parameter such as a clinical condition in need of treatment. The correlation steps used in the methods of this invention may involve several steps of linear regression analysis.

The concentration of a given compound is preferably calculated by using a calibration equation derived from a statistical analysis, for example but not limited to a least-squares best fit, of a plot of the values of concentration of a calibration set of samples of the compound—which can be determined using the method described herein versus the values of the concentration of the calibration set measured by a different method (e.g., directly). Any known method for determining the concentration of one or more compounds may be used.

One technique that may be employed is the utilization of first or second derivate spectra and the intensity of dominant bands in this spectra (such as, for example, 1057, 1466, 1381 cm⁻¹ for cholesterol or (1740, 1466, 1381, 1170 cm⁻¹ for cholesterol ester). These bands in primary spectra or second degree spectra may be normalized to protein content, such as amide bands at 1550 cm⁻¹, by dividing peak heights of second derivative cholesterol, for example at 1057 cm⁻¹, to the height of amide II protein @1550 cm⁻¹. This can provide information in a form which can be related to Cholesterol(mg)/Protein(mg). This can create a means to more accurately compare measurements between different subjects. Similar techniques can be applied to the other species of interest: the cholesterol species, free fatty acids, triglycerides, lipids, phospholipids, etc.

In some embodiments, there is provided a method that identifies a clinical condition in a human or animal by correlating the concentration of a measured compound in the skin of the human or animal to a clinical condition in need of treatment using a correlation algorithm. In this case, the correlation algorithm determines the correlation between the concentration of the compound and a positive result from a medical test that screens for a particular clinical condition.

In some embodiments, there is provided a method that identifies the risk of a clinical condition in a human or animal by correlating the concentration of a measured compound in the skin of a human or an animal to the risk of a clinical condition in need of treatment using a correlation algorithm. In this case, the correlation algorithm determines the correlation of the concentration of the compound with respect to a result from a medical test that screens for a particular clinical condition. This comparison can result in a positive correlation.

Examples of the medical test mentioned above include coronary angiography, coronary calcium load by CT scanning, stress test, intravascular coronary ultrasound, flow-mediated brachial vasoactivity, and carotid sonography.

In some embodiments, a method can comprise any of the following steps, in any combination or order: 1) preparing (e.g., scraping or cleaning) tissue; 2) contacting a probe to tissue (or placing a probe within a given distance of tissue); 3) irradiating tissue (e.g. skin) with infrared (e.g., mid-infrared) radiation; 4) exciting molecules of a species in the tissue; 5) receiving information from those molecules; and/or 6) correlating the measured concentration to a clinical condition (or, e.g., a propensity for a clinical condition).

Methods and processes described above may be embodied in, and fully automated via, software code modules executed by one or more general purpose computers. The code modules may be stored in any type of computer-readable medium or other computer storage device. Some or all of the methods may alternatively be embodied in specialized computer hardware. The collected user feedback data (e.g., accept/rejection actions and associated metadata) can be stored in any type of computer data repository, such as relational databases and/or flat files systems.

Reference throughout this specification to “some embodiments” or “an embodiment” means that a particular feature, structure or characteristic described in connection with the embodiment is included in at least some embodiments. Thus, appearances of the phrases “in some embodiments” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

In the above description of embodiments, various features of the inventions are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single foregoing disclosed embodiment.

A number of applications, publications and external documents are incorporated by reference herein. Any conflict or contradiction between a statement in the bodily text of this specification and a statement in any of the incorporated documents is to be resolved in favor of the statement in the bodily text.

Although the invention(s) presented herein have been disclosed in the context of certain preferred embodiments and examples, it will be understood by those skilled in the art that the invention(s) extend beyond the specifically disclosed embodiments to other alternative embodiments and/or uses of the invention(s) and obvious modifications and equivalents thereof. Thus, it is intended that the scope of the invention(s) herein disclosed should not be limited by the particular embodiments described above.

1. A method of accurately measuring body cholesterol levels without inaccuracies related to recent diet, the method comprising:
irradiating skin tissue with mid-infrared radiation to excite lipid molecules within superficial layers of the tissue;
measuring resulting mid-infrared radiation;
comparing the measured radiation to stored reference data to obtain correlation information; and
using the correlation information to determine body lipid levels without inaccuracies related to recent diet.

2. The method of claim 1, wherein measuring resulting mid-infrared radiation comprises measuring reflected mid-infrared radiation.

3. The method of claim 1, wherein measuring resulting mid-infrared radiation comprises measuring transmitted mid-infrared radiation.

4. The method of claim 1, wherein irradiating skin tissue with mid-infrared radiation to excite lipid molecules within superficial layers of the tissue comprises irradiating skin tissue with mid-infrared radiation to excite all cholesterol molecules within superficial layers of the tissue.

5. The method of claim 1, wherein irradiating skin tissue with mid-infrared radiation to excite lipid molecules within superficial layers of the tissue comprises irradiating skin tissue with mid-infrared radiation to excite esterified cholesterol molecules within superficial layers of the tissue.

6. The method of claim 1, wherein irradiating skin tissue with mid-infrared radiation to excite lipid molecules within superficial layers of the tissue comprises irradiating skin tissue with mid-infrared radiation to excite free cholesterol molecules within superficial layers of the tissue.

7. The method of claim 1, wherein irradiating skin tissue with mid-infrared radiation to excite lipid molecules within superficial layers of the tissue comprises irradiating skin tissue with mid-infrared radiation to excite free fatty acids within superficial layers of the tissue.

8. The method of claim 1, wherein irradiating skin tissue with mid-infrared radiation to excite lipid molecules within superficial layers of the tissue comprises irradiating skin tissue with mid-infrared radiation to excite ceramides within superficial layers of the tissue.

9. The method of claim 1, further comprising using a lipid level in a body to assess a disease.

10. The method of claim 9, wherein using the lipid level to assess a disease comprises using body cholesterol to diagnose psoriasis.

11. The method of claim 9, wherein using the lipid level to assess a disease comprises using esterified cholesterol to diagnose psoriasis.

12. The method of claim 9, wherein using the lipid level to assess a disease comprises using a ceramide to diagnose psoriasis.

13. The method of claim 9, wherein using the lipid level to assess a disease comprises using a free fatty acid level to diagnose psoriasis.

14. The method of claim 9, wherein using the lipid to assess a disease comprises using body cholesterol to assess risk of cardiovascular disease.

15. A method of determining a living body's long-term deposition pattern of a compound, the method comprising:
placing skin of a living body against a receptor;
directing mid-infrared electromagnetic radiation onto the skin of the living body;
quantifying the electromagnetic radiation that is reflected from the skin to obtain a reflected amount;
using a calibration equation to calculate the concentration of a compound from the reflected amount; and
using the concentration of the compound to evaluate risk of a clinical condition.

16. The method of claim 15, wherein using a calibration equation to calculate the concentration of a compound from the reflected amount comprises using a least-squares best fit statistical comparison.

17. The method of claim 15, wherein using the concentration of the compound to evaluate risk of a clinical condition comprises using the concentration of the compound to evaluate risk of cardiovascular disease.

18. The method of claim 15, wherein using a calibration equation to calculate the concentration of a compound from the reflected amount comprises using a calibration equation to calculate the concentration of a lipid from the reflected amount

19. The method of claim 18, wherein using a calibration equation to calculate the concentration of a lipid from the reflected amount comprises using a calibration equation to calculate the concentration of cholesterol from the reflected amount.

20. The method of claim 15, wherein using the concentration of the compound to evaluate risk of a clinical condition comprises using an algorithm that indicates correlation between amount of the compound and presence of a medical condition.

21. The method of claim 15, wherein using the concentration of the compound to evaluate risk of a clinical condition comprises using an algorithm that indicates correlation between amount of the compound and severity of a medical condition.

22. The method of claim 15, wherein using the concentration of the compound to evaluate risk of a clinical condition comprises using an algorithm that indicates correlation between amount of the compound and risk of a developing a medical condition.

23. The method of claim 15, wherein using the concentration of the compound to evaluate risk of a clinical condition comprises using an algorithm that indicates correlation between amount of the compound and a prediction of success of a treatment for a medical condition.

24. The method of claim 15, wherein using the concentration of the compound to evaluate risk of a clinical condition comprises using an algorithm that indicates correlation between amount of the compound and a documentation of success of a treatment for a medical condition.

25. A method of testing for a type of cholesterol to diagnose a clinical condition, the method comprising:
preparing tissue;
placing a probe in proximity to tissue;
irradiating tissue with infrared radiation, thereby exciting molecules of a species in the tissue;
collecting resulting information from those molecules to determine a concentration of molecules of that species within the tissue; and
correlating the concentration to a clinical condition.

26. The method of claim 25, wherein preparing comprises scraping.

27. The method of claim 25, wherein preparing comprises cleaning.

28. The method of claim 25, wherein placing a probe in proximity to tissue comprises contacting the probe to the tissue.

29. The method of claim 25, wherein preparing tissue comprises preparing skin tissue and wherein irradiating tissue comprises irradiating the skin tissue.

30. The method of claim 25, wherein irradiating tissue with infrared radiation comprises irradiating tissue with mid-infrared radiation.

31. The method of claim 25, wherein correlating the concentration to a clinical condition comprises correlating the concentration to a propensity for a clinical condition.

32. The method of claim 25, wherein the tissue is skin, further comprising all of the same steps performed at least a second time on a second tissue portion located deeper in a subject's skin.

33. The method of claim 25, wherein the tissue is skin, further comprising all of the same steps performed at least a second time on a second tissue portion located on the same skin level.

34. The method of claim 33, wherein the steps are performed one time on skin affected by a dermatological condition and another time on skin unaffected by the dermatological condition.

35-75. (canceled)
